(19) United States
(12) Patent Application Publication
(10) Pub. No.: US 2013/0256932 A1
(43) Pub. Date: Oct. 3, 2013

(54) METHOD FOR PREPARING A HEMISPHERE-SHAPED DOSAGE FORM CONTAINING DRUG AND APPLICATIONS THEREOF

(71) Applicant: Instrument Technology Research Center, National Applied Research Laboratories, Hsinchu (TW)

(72) Inventors: Yung-Sheng LIN, Hsinchu (TW); Chun-Ting LIN, Hsinchu (TW); Keng-Shiang HUANG, Hsinchu (TW); Chih-Hui YANG, Hsinchu (TW); Sheng-Yi HSIAO, Hsinchu (TW); Chih-Cheng HUANG, Hsinchu (TW); Shih-Feng TSENG, Hsinchu (TW)

(73) Assignee: Instrument Technology Research Center, National Applied Research Laboratories, Hsinchu (TW)

(21) Appl. No.: 13/903,654

(22) Filed: May 28, 2013

Related U.S. Application Data
(63) Continuation-in-part of application No. 12/763,408, filed on Apr. 20, 2010, now abandoned.

(51) Int. Cl. A61K 9/14

(52) U.S. Cl. CPC A61K 9/14
USPC 264/13; 264/5

(57) ABSTRACT

A method for preparing a hemisphere-shaped dosage form containing drug. A high molecular weight solution containing the drug is prepared, and the solution is then dropped onto a base material. The interface phenomenon between the solution and different base materials causes the solution to form a hemisphere-shaped liquid drop dosage. After solidifying by cross-link or evaporation, a hemisphere-shaped dosage form containing drug is obtained. The advantages of the preparation method are a simple and fast process, and simple operation. Applications of the preparation method to prepare a hemisphere-shaped dosage form containing drug are also provide.

### CROSS REFERENCE TO RELATED APPLICATIONS

This application is a continuation-in-part of co-pending application Ser. No. 12/763,408 filed on 20 Apr. 2010, and for which priority is claimed under 35 U.S.C. §120; the entire contents of which are hereby incorporated by reference.

### BACKGROUND OF THE INVENTION

1. Field of the Invention

The present invention is related to a novel method of preparing a drug delivery form, in particular, a method of preparing a micro-particle drug in hemisphere-shaped dosage form, and applications thereof

2. Description of the Prior Art

Recent studies pertain to Drug Delivery Systems (DDS), i.e., preventing overdosage, side effects, promoting safe and effective ways for micro-particle drug delivery, and providing the necessary minimal amount of a drug dose to the necessary part in the necessary time. Most studies focus on development of Drug Delivery Systems for 1) slowly dissolving a drug in the body, and/or 2) delivering an adequate amount of the drug by the blood stream to the nidus for treatment. These studies relate to the improvement of a drug's characteristics or the development of materials, methods or structures for embedding the drug. In 1998, Ozdemir and Karatas pointed out that the shape or geometry of the drug could affect the release rate thereof. They also found that a water-soluble drug or slightly oil-soluble drug in a hemisphere shape achieves a certain release rate in comparison with that of the drug in other shapes, such as a cylinder, slab, or biconvex form. In “Analysis of nano drug carriers towards optimum release rate” (Journal of Medical Engineering & Technology, Vol. 31, No. 4, pp. 243-252 to Ng et al. indicate that a drug in hemisphere-shaped dosage form could reach a zero-order drug release rate in comparison to tetrahedron, cylinder, sphere, cuboid, and octahedron shapes. However, the art remains silent with regard to preparing and/or applying hemisphere-shaped dosage forms for drug delivery.

Besides improving the release rate of a drug, the development of a new dosage form for drug delivery can reduce the research and development costs of a drug greatly, thus giving the product itself a high added-value. In comparison with development of a new drug, the risk of developing a new dosage form is much lower. Based on the importance of developing new dosage forms for drug delivery, the inventors of the present invention provide a method for preparing a micro-particle drug in hemisphere-shaped dosage form and applications thereof.

### SUMMARY OF THE INVENTION

The object of the present invention is to provide a novel method for preparing a hemisphere-shaped dosage form containing drug.

Another purpose of the present invention is to provide applications of the method for preparing a hemisphere-shaped dosage form containing drug. In order to accomplish the purposes listed above, the method of the present invention comprises preparing a high molecular weight solution containing the drug, wherein the high molecular weight solution has a molecular weight of 5,000˜500,000 and comprises at least one of alginate, chitin, chitosan, poly lactic acid (PLA), or poly glycolic acid (PGA), and then dropping this solution onto a base material having a predefined surface to produce a hemisphere-shaped liquid drop dosage. The predefined surface is defined by photolithography to produce circles of 0.5-1000 μm diameter on the base material. The space between the circles is treated with C4F8 gas, and the space becomes hydrophobic. The interface phenomenon between the high molecular weight solution and the base material causes the high molecular weight solution to form a hemisphere-shaped liquid drop dosage containing drug within the circle on the base material, wherein the base material is selected from silicon, metal, carbon, cupper, silver, platinum or gold. After a solidifying and separating process, the hemisphere-shaped dosage form is obtained, wherein the solidifying process comprises at least one of cross-link and evaporation, and the hemisphere-shaped dosage form contains drug, high molecular weight ingredient, and water, and thus could can achieve a zero order drug release rate in a patient's body. The size of the hemisphere-shaped dosage form is controllable and determined by the size of the circle on the base material. The size of the hemisphere dosage form manufactured by the method is 0.5-1000 μm diameter.

A second method for preparing a hemisphere-shaped dosage form containing drug comprises preparing a high molecular weight solution containing the drug, wherein the high molecular weight solution has a molecular weight of 5,000˜500,000 and comprises at least one of alginate, chitin, chitosan, poly lactic acid (PLA), or poly glycolic acid (PGA), and a base material with a predefined surface, wherein the predefined surface is defined by photolithography to produce circles of 0.5-1000 μm diameter on the base material. The space between the circles is treated with C4F8 gas, and the space becomes hydrophobic. The base material with a predefined surface is then dipped into the high molecular weight solution. Next, the base material is removed from the solution, and interface phenomenon between the solution and base material causes the solution to form hemisphere-shaped liquid drop dosage containing drug within the circle on the base material. The base material is selected from silicon, metal, carbon, cupper, silver, platinum, or gold. After a solidifying and separating process, the hemisphere-shaped dosage form containing drug is obtained, wherein the solidifying process comprises at least one of cross-link and evaporation. The hemisphere-shaped dosage form contains drug, high molecular weight ingredient and water, and thus can achieve a zero-order drug release rate in a patient's body. The size of the hemisphere-shaped dosage form is controllable and determined by the size of the circle on the base material. The size of the hemisphere-shaped dosage form manufactured by the method is 0.5-1000 μm diameter.

A third method for preparing a hemisphere-shaped dosage form containing drug comprises preparing a high molecular weight solution containing drug, wherein the high molecular weight solution has a molecular weight of 5,000˜500,000 and comprises at least one of alginate, chitin, chitosan, poly lactic acid (PLA), or poly glycolic acid (PGA). The solution is injected onto an untreated base material by inkjet printing (IJP) with a nozzle of 30-100 μm diameter, to form hemisphere-shaped liquid drop dosage on the surface of the base material. The base material is selected from silicon, metal, carbon, cupper, silver, platinum or gold. After a solidifying and separating process, the hemisphere-shaped dosage form containing drug is obtained, wherein the solidifying process comprises at least one of cross-link and evaporation. The hemisphere-shaped dosage form contains drug, high molecular weight ingredient, and water, and thus can achieve a zero-order drug release rate in a patient's body. The size of the hemisphere-shaped dosage form is controllable and determined by the size of the nozzle. The size of the hemisphere-shaped dosage form manufactured by the method is 30-100 μm diameter.

These features and advantages of the present invention will be fully understood and appreciated from the following detailed description and the accompanying drawings.

### BRIEF DESCRIPTION OF THE DRAWINGS

FIGS. 1A-1C demonstrate the preparation of a hemisphere-shaped dosage form containing drug. FIG. 1A shows a hemisphere-shaped liquid drop dosage 1 containing drug on a base material 2. FIG. 1B shows the hemisphere-shaped dosage form 3 containing drug after solidifying. FIG. 1C shows the separation of the hemisphere-shaped dosage form 3 from the base material 2 to retrieve the hemisphere-shaped dosage form containing drug.

FIGS. 2A and 2B show the operation of producing hemisphere-shaped dosage form by dropping a high molecular weight solution onto a predefined base material.

FIG. 3 shows a high molecular weight solution being injected by an injecting printing (IJP).

### DETAILED DESCRIPTION OF THE PREFERRED EMBODIMENTS

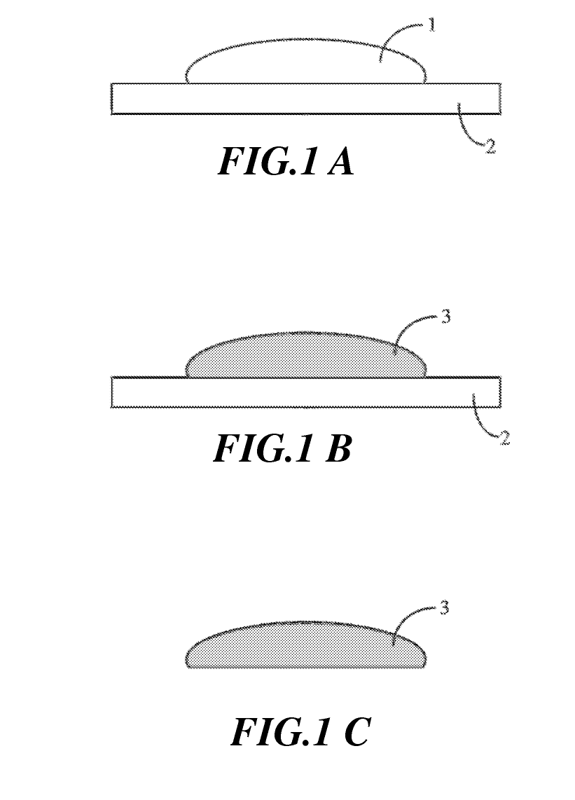

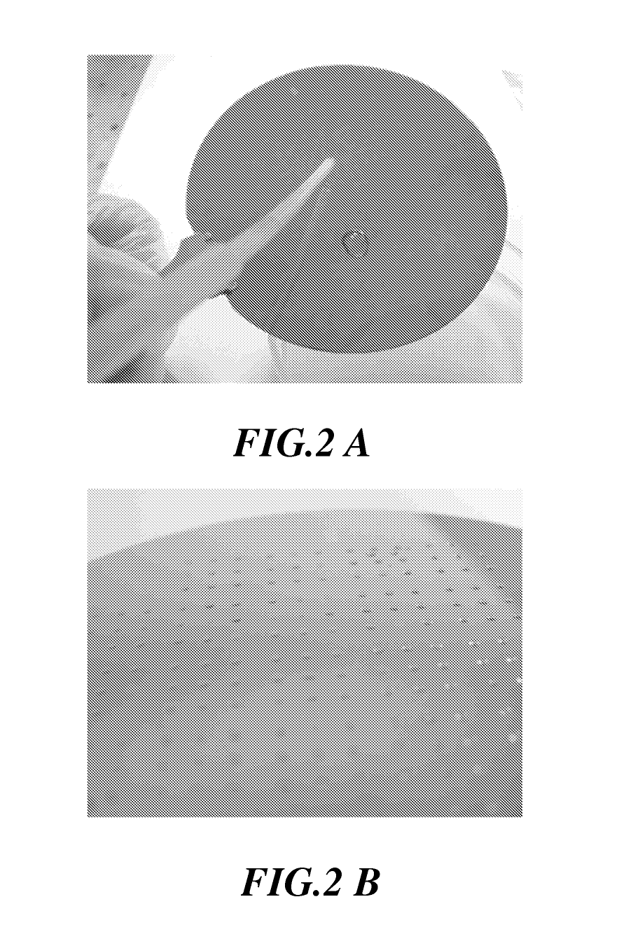

The present invention provides a novel method for preparation of a hemisphere-shaped dosage form containing drug. As shown in FIGS. 1A-1C, 2A, and 2B, the method comprises preparing a high molecular weight solution containing a drug, and dropping the solution onto the base material 2 with a predefined surface to produce a hemisphere-shaped liquid drop dosage 1, and injecting the solution onto the base material 2 or dipping the base material 2 with a predefined surface into the solution. After solidifying and separating, a hemisphere-shaped dosage form 3 containing drug is obtained.

Solidifying steps include, but are not limited to, cross-link, evaporation, and so on. Separation steps include, but are not limited to, methods of physical or chemical separation and any separation methods that are suitable to be used in this invention.

The drugs include, but are not limited to, any drugs that are suitable to be used in the preparation of this invention.

The high molecular weight ingredient of the high molecular solutions include, but are not limited to, alginate, chitin, chitosan, poly lactic acid (PLA), poly glycolic acid (PGA), and any high molecular ingredients that are suitable to prepare a high molecular weight solution of this invention

The high molecular weight solution has a molecular weight of 5,000˜500,000.

The surface of the base material is predefined by photolithography to produce circles of 0.5-1000 μm diameter on the surface of the base material.

In one preferred embodiment, the high molecular weight solution contains at least 1%˜5% drug, 1%˜40% alginate, and 55%˜98% water. Furthermore, the high-molecular-weight solution is cross linked by an effective amount of calcium ion (e.g. a compound or composition that contains calcium), and a solidified hemisphere-shaped dosage form containing alginate is prepared.

The “effective amount” of a calcium ion (e.g. a compound or composition that contains calcium) means that the amount or concentration of calcium ion is enough to trigger cross-linking and solidifying of the high molecular weight solution.

In another preferred embodiment, the high molecular weight solution contains at least 0.05%˜20% drug, 1.95%˜49.95% PLA, and 50%˜98% organic solvent, wherein the organic solvent includes, but is not limited to, methylene chloride, ethyl acetate, and acetone. Furthermore, the solidified hemisphere-shaped dosage form containing PLA is prepared after complete evaporation of the organic solvent.

The hemisphere-shaped dosage form containing drug prepared by the present invention has a diameter of 0.5˜1000 μm.

The invention will be illustrated with the examples as follows, without the intention that the invention is limited thereto. The substance or material herein are easily obtained, and the source of materials is not limited to following examples.

### EXAMPLE 1

Preparation of Hemisphere-Shaped Dosage Form with Alginate

1. Reference is made to FIGS. 1A-1C, 2A, and 2B. An aqueous solution containing drug and alginate is prepared, and the solution is then dropped onto the a base material having a predefined surface to form a hemisphere-shaped liquid drop dosage. A solution containing calcium is then added into the hemisphere-shaped liquid drop dosage to trigger the cross-link reaction (i.e., the solidification step). The alginate is gelatinous and the hemisphere-shaped dosage form is then formed. The base material is then removed to obtain a hemisphere-shaped dosage form containing drug.

2. An aqueous solution containing drug and alginate is prepared, and a base material having a predefined surface is then dipped into the solution. Next, the base material is removed from the solution and a hemisphere-shaped liquid drop dosage is formed on the base material. A solution containing calcium is then added into the hemisphere-shaped liquid drop dosage to trigger the cross-link reaction (i.e., the solidification step). The alginate is gelatinous and the hemisphere-shaped dosage form is then formed. The base material is then removed to obtain the hemisphere-shaped dosage form containing drug.

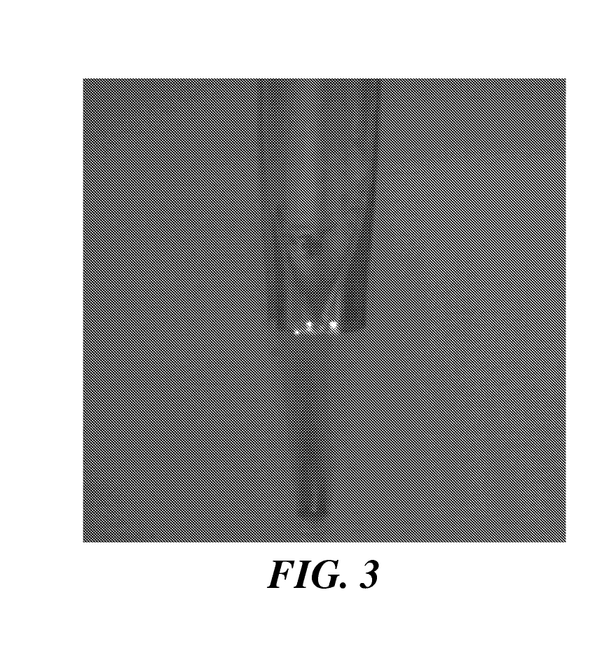

3. An aqueous solution containing drug and alginate is prepared, and the solution is then injected onto a base material by injecting printing (IJP) (FIG. 3) to form a hemisphere-shaped liquid drop dosage. Next, a solution containing calcium is added into the hemisphere-shaped liquid drop dosage to trigger the cross-link reaction (i.e., the solidification step). The alginate is gelatinous and the hemisphere-shaped dosage form is then formed. The base material is then removed to obtain the hemisphere-shaped dosage form containing drug.

### EXAMPLE 2

Preparation of Micro-particle of PLA in Hemi-sphere Dosage Form

1. Reference is made to FIGS. 1A-1C, 2A, and 2B. A methylene chloride solution containing drug and PLA is prepared, and the solution is then dropped onto a base material having a predefined surface. A hemisphere-shaped liquid drop dosage containing drug and PLA is formed and then stewed for a while to let the organic solvent evaporate. The hemisphere-shaped liquid drop dosage is semi-solidified and not mobile. The base material is then removed to obtain the semi-solidified hemisphere-shaped dosage form containing drug and PLA. Finally, the solidified hemisphere-shaped dosage form containing drug and PLA is obtained by further evaporating to remove any remaining organic solvent completely. The solidified hemisphere-shaped dosage form contains 0.1%˜40% drug and 60%˜99.9% PLA.

2. A methylene chloride solution containing drug and PLA is prepared, and a base material having a predefined surface is dipped into the solution. The base material is then removed from the solution and a hemisphere-shaped liquid drop dosage containing drug and PLA is formed on the base material. The hemisphere-shaped liquid drop dosage containing drug and PLA is formed and then stewed for a while to let the organic solvent evaporate. The hemisphere-shaped liquid drop dosage is semi-solidified and not mobile. The base material is then removed to obtain the semi-solidified hemisphere-shaped dosage form containing drug and PLA. Finally, the solidified hemisphere-shaped dosage form containing drug and PLA is obtained by further evaporating to remove any remaining organic solvent completely. The solidified hemisphere-shaped dosage form contains 0.1%˜40% drug and 60%˜99.9% PLA.

3. A methylene chloride solution containing drug and PLA is prepared, and the solution is then injected onto the base material by IJP (FIG. 3). A hemisphere-shaped liquid drop dosage containing drug and PLA is formed on the base material and then stewed for a while to let the organic solvent evaporate. The hemisphere-shaped liquid drop dosage is semi-solidified and not mobile. The base material is then removed to obtain the semi-solidified hemisphere-shaped dosage form containing drug and PLA. Finally, the solidified hemisphere-shaped dosage form containing drug and PLA is obtained by further evaporating to remove any remaining organic solvent completely. The solidified hemisphere-shaped dosage form contains 0.1%˜40% drug and 60%˜99.9% PLA.

Compared with the current state of the arts and products, the present invention provides a preparation method and its applications to produce a hemisphere-shaped dosage form containing drug. The present invention contains the following advantages:

1. The method of present invention to prepare hemisphere-shaped dosage form is novel and its procedure is simple, fast, and easily operated.

2. The hemisphere-shaped dosage form containing drug prepared by the method of the present invention has a different drug release rate from that of drugs with a spherical, cylindrical, and slab shapes, and the method of the present invention can be applied to any suitable drug.

3. Any drug that is suitable to be prepared by the present invention could reach a zero-order drug release rate in a patient's body after being produced by this method of the invention.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. A method for preparing a hemisphere-shaped dosage form containing drug, comprising:
dropping a high molecular weight solution containing drug onto a base material with predefined surface to produce a hemisphere-shaped liquid drop dosage, wherein the high molecular weight solution has a molecular weight of 5,000˜500,000 and comprises at least one of alginate, chitin, chitosan, poly lactic acid (PLA), or poly glycolic acid (PGA); and
performing a solidifying process to obtain a solidified hemisphere-shaped dosage form containing the drug,
wherein the solidifying processes comprises at least one of cross-link and evaporation,

2. The method as recited in claim 1, wherein the predefined surface is predefined by photolithography to produce circles of 0.5-1000 μm diameter on the surface of the base material.

3. The method as recited in claim 1, wherein the hemisphere-shaped dosage form has a diameter of 0.5-1000 μm.

4. The method as recited in claim 1, wherein the high molecular weight solution is aqueous and comprises at least 1%˜5% drug, 1%˜40% alginate, and 55%˜98% water, wherein the solidifying process is preceded by adding an effective amount of a calcium ion solution, compound, or composition that contains calcium into the hemisphere-shaped liquid drop dosage to trigger a cross-link reaction, and a solidified hemisphere-shaped dosage form containing alginate is obtained.

5. The method as recited in claim 1, wherein the high molecular weight solution comprises 0.05%˜20% drug, 1.95%˜49.95% poly lactic acid (PLA), and 50%˜98% organic solvent, wherein the organic solvent comprises at least one of methylene chloride, ethyl acetate, and acetone.

6. The method as recited in claim 1, wherein the high molecular weight solution containing drug comprises 0.05%˜20% drug, 1.95%˜49.95% poly lactic acid (PLA), and 50%˜98% methylene chloride, and the solidified hemisphere-shaped dosage form comprises 0.1%˜40% drug and 60%˜99.9% PLA after the organic solvent is evaporated completely.

7. A method for preparing a hemisphere-shaped dosage form containing drug, comprising:
dipping a base material with a predefined surface into a high molecular weight solution containing drug, wherein the high molecular weight solution has a molecular weight of 5,000˜500,000 and comprises at least one of alginate, chitin, chitosan, poly lactic acid (PLA), or poly glycolic acid (PGA); and
removing the base material from the high molecular weight solution to produce a hemisphere-shaped liquid drop dosage; and
performing a solidifying process to obtain a solidified hemisphere-shaped dosage form containing the drug, wherein the solidifying processes comprises at least one of cross-link and evaporation.

8. The method as recited in claim 7, wherein the predefined surface is predefined by photolithography to produce circles of 0.5˜1000 μm diameter on the surface of the base material.

9. The method as recited in claim 7, wherein the hemisphere-shaped dosage form has a diameter of 0.5-1000 μm.

10. The method as recited in claim 7, Wherein the high molecular weight solution is aqueous and comprises at least 1%˜5% drug, 1%˜40% alginate, and 55%˜98% water, wherein the solidifying process is preceded by adding an effective amount of calcium ion solution, compound, or composition that contains calcium into the hemisphere-shaped liquid drop dosage to trigger a cross-link reaction, and a solidified hemisphere-shaped dosage form containing alginate is obtained.

11. The method as recited in claim 7, wherein the high molecular weight solution comprises 0.05%˜20% drug, 1.95%˜49,95% poly lactic acid (PLA), and 50%˜98% organic solvent, wherein the organic solvent comprises at least one of methylene chloride, ethyl acetate, and acetone.

12. The method as recited in claim 7, wherein the high molecular weight solution containing drug comprises 0.05%˜20% drug, 1.95%˜49.95% poly lactic acid (PIA), and 50%˜98% methylene chloride, and the solidified hemisphere-shaped dosage form comprises 0.1%˜40% drug and 60%˜4)9.9% PLA after the organic solvent is evaporated completely.

13. A method for preparing a hemisphere-shaped dosage form containing drug, comprising: injecting a high molecular weight solution by inkjet printing (UP) onto a base material to produce a hemisphere-shaped liquid drop dosage, wherein the high molecular weight solution has a molecular weight of 5,000˜500,000 and comprises at least one of alginate, chitin, chitosan, poly lactic acid (PLA), or poly glycolic acid (PGA); and
performing a solidifying process to obtain a solidified hemisphere-shaped dosage form containing the drug, wherein the solidifying processes comprises at least one of cross-link and evaporation.

14. The method as recited in claim 13, wherein the UP has a nozzle of 30-100 μm in diameter.

15. The method as recited in claim 13, wherein the hemisphere-shaped dosage form has a diameter of 30-100 μm.

16. The method as recited in claim 13, wherein the high molecular weight solution is aqueous and comprises at least 1%˜% drug, 1%-40% alginate, and 55%˜98% water, wherein the solidifying process is preceded by adding an effective amount of a calcium ion solution, compound, or composition that contains calcium into the hemisphere shaped aqueous liquid drop dosage to trigger a cross-link reaction, and a solidified hemisphere shaped dosage form containing alginate is obtained.

17. The method as recited in claim 13, wherein the high molecular weight solution comprises 0.05%˜20% drug, 1.95%˜49.95% poly lactic acid (PLA), and 50%˜98% organic solvent, wherein the organic solvent comprises at least one of methylene chloride, ethyl acetate, and acetone.

18. The method as recited in claim 13, wherein the high molecular weight solution containing drug comprises 0.05%˜20% drug, 1.95%˜49.95% poly lactic acid (PLA), and 50%˜98% methylene chloride, and the solidified hemisphere-shaped dosage form comprises 0.1%˜40% drug and 60%˜99.9% PLA after the organic solvent is evaporated completely.
